(19) United States
(12) Patent Application Publication
(10) Pub. No.: US 2026/0354675 A1
(43) Pub. Date: Nov. 12, 2026

(54) DIAGNOSTIC DEVICE FOR COLONOSCOPY

(71) Applicant: Università degli Studi di Padova, Padova (PD) (IT)

(72) Inventor: Lino Polese, Venezia (VE) (IT)

(21) Appl. No.: 19/137,157

(22) PCT Filed: Dec. 1, 2023
(86) PCT No.: PCT/IB2023/062115

§ 371 Date: Jun. 9, 2025

(30) Foreign Application Priority Data
Dec. 7, 2022 (IT) 102022000025215

(51) Int. Cl. A61B 1/31; A61B 1/00; A61B 1/32

(52) U.S. Cl. CPC A61B 1/31; A61B 1/00137; A61B 1/32

(57) ABSTRACT

The present disclosure relates to an anoscope (100) for colonoscopy comprising a main body (10) extending along a main axis of development (A) between a first end portion (11) and a second end portion (12), wherein said second end portion (12) is configured, in use, to be at least partially inserted into a patient's body. The main body (10) defines a through cavity (13) along said main axis of development (A) between the first end portion (11) and the second end portion (12). The main body (10) includes a first surface, or outer surface, configured, in use, to face an anal canal of a patient, and a second surface, opposite to the first surface, or inner surface, and a plurality of openings (21, 31, 41), each configured to connect said first surface, or outer surface, to said second surface, or inner surface, of said main body (10). The anoscope (100) further comprises a hermetic closure element (50) configured to hermetically close said through cavity (13) at said first end portion (11) of said main body (10).

This disclosure pertains generally to diagnostic devices. More specifically, this disclosure relates to a diagnostic device for colonoscopy.

Colonoscopy is a crucial diagnostic examination for colon cancer screening. It is performed using a flexible endoscope and enables early diagnosis of colon tumors. Additionally, it allows for the removal of preneoplastic lesions, such as adenomas and early tumors in the colon and rectum, using techniques like polypectomy.

Colonoscopy is also employed in cases of acute and chronic inflammatory bowel diseases, as well as for lower gastrointestinal bleeding. During colonoscopy, the endoscope is introduced through the anal canal, and with the insufflation of air or CO₂, it distends the intestinal walls to adequately visualize the mucosal surface and luminal contents.

Through this examination, it is possible to visualize the terminal ileum, the colon, including the cecum, ascending colon, transverse colon, descending colon, sigmoid colon, and rectum.

However, conventional colonoscopy usually does not provide adequate visualization of the anal canal. This is because the anus constricts around the colonoscope during instrument passage due to sphincter contractions. As a result, the lack of separation between the walls of the anus and the colonoscope prevents proper visualization of the anal canal. While the walls of the colonic and rectal lumen expand due to insufflation (with air or CO₂), the contraction of the anus hinders its distension.

Visualizing the anus during colonoscopy would enable the diagnosis of anal canal pathologies such as anal fissures, hemorrhoids, anal tumors, and polyps. Furthermore, visualizing the anorectal passage would help recognize polyps or tumors located in the most distal part of the rectum.

In order to improve the examination, and specifically enhance the visualization of the anorectal passage, colonoscopy involves the retroversion maneuver. This maneuver entails inserting the endoscope into the rectal ampulla of a patient and then bending it like an umbrella handle to directly view the distal rectal segment above the anus in the opposite direction, i.e., from the rectal ampulla.

This maneuver, performed with the colonoscope, is characterized by certain limitations.

Firstly, it is particularly risky as it can potentially damage the patient's rectum.

Secondly, complete visualization of the anus is still not possible, especially in its distal segment where it surrounds the endoscope.

In conclusion, known colonoscopes do not allow for a complete visualization of the anus during colonoscopy.

The present disclosure aims to provide an anoscope that overcomes the aforementioned drawbacks associated with the known technique and/or achieves additional advantages.

This is accomplished through an anoscope and a diagnostic device as defined in the respective independent claims. Secondary features and specific embodiments of the subject matter of this disclosure are defined in the corresponding dependent claims.

The present disclosure begins with the acknowledgment by the author that during a colonoscopy, complete visualization of the anus is not possible.

The anoscope for colonoscopy according to the present disclosure comprises a main body that extends along a main axis of development between a first end portion and a second end portion, configured to be at least partially inserted, in use, into a patient's body. The main body also defines a through cavity along the main axis of development, wherein this through cavity extends between the first and second end portions of the main body.

The anoscope according to the present disclosure further comprises a hermetic closure element configured to hermetically close the through cavity at the first end portion of the main body.

The main body of the anoscope according to the present disclosure also includes a first surface, or outer surface, configured, in use, to face the anal canal of a patient, and a second surface, or inner surface, opposite to the first surface. In other words, the second surface, or inner surface, is facing the through cavity defined by the main body.

According to the present disclosure, the main body comprises a plurality of openings, preferably through openings, each configured to connect the first surface to the second surface of the main body.

Consequently, advantageously, through these openings, direct visualization of the anal canal of a patient is possible. In other words, unlike known anoscopes, the anoscope according to the present disclosure includes a main body with a plurality of openings, windows, or holes that directly connect the outer surface and the inner surface of the main body itself, allowing for the visualization of the anal canal. These openings or windows adequately expose at least a portion of the surface of the anal canal to an endoscope so that it can be directly viewed.

Preferably, according to an aspect of the present disclosure, each opening in the main body is formed by protruding elements extending from the first end portion towards the second end portion of the main body. In other words, each opening is formed between two consecutive protruding elements, which can be, according to a preferred aspect of the present disclosure, elongated bodies extending from the first end portion of the main body. Stated differently, the main body of the anoscope according to the present disclosure is a fenestrated main body, or equipped with openings or windows, allowing for the visualization of the anal canal of a patient.

For example, the protruding elements may be arranged circumferentially to form the through cavity, and each opening may be defined between two consecutive protruding elements along a circumferential direction.

According to a preferred aspect of the present disclosure, three protruding elements may be provided, defining three openings in the main body.

In a further preferred aspect of the present disclosure, the hermetic closure element of the anoscope is made of elastic material, preferably silicone.

Additionally, preferably, this hermetic closure element includes a through hole configured, in use, for the passage of an endoscope.

Consequently, this hermetic closure element advantageously prevents or avoids the leakage of insufflated gas during the endoscopic examination on one hand, and on the other hand, allows the passage of an endoscope through it and into the through cavity of the main body.

In another preferred aspect of the present disclosure, the anoscope further comprises an actuation device. This actuation device is associated with the first end portion of the main body and is configured, in use, to allow rotation of the main body around the main axis of development. In this way, advantageously, an operator can rotate the anoscope during colonoscopy, thereby changing the position of the windows or openings of the main body relative to the anal canal of a patient.

Preferably, according to a preferred aspect, the anoscope according to the present disclosure further includes a dilator device configured to be removably inserted into the through cavity of the main body. This makes the insertion of the anoscope less traumatic for a patient.

Furthermore, the present disclosure further encompasses a diagnostic device comprising an anoscope designed to accommodate an endoscope.

Additional advantages, features, and methods of use of the subject matter of the present disclosure will become apparent from the following detailed description of its embodiments, presented for illustrative purposes and not limiting.

It is, however, evident how each embodiment of the subject matter of the present disclosure may have one or more of the above-listed advantages; nonetheless, it is not required that each embodiment simultaneously possesses all the listed advantages.

Reference will be made to the figures of the attached drawings, in which:

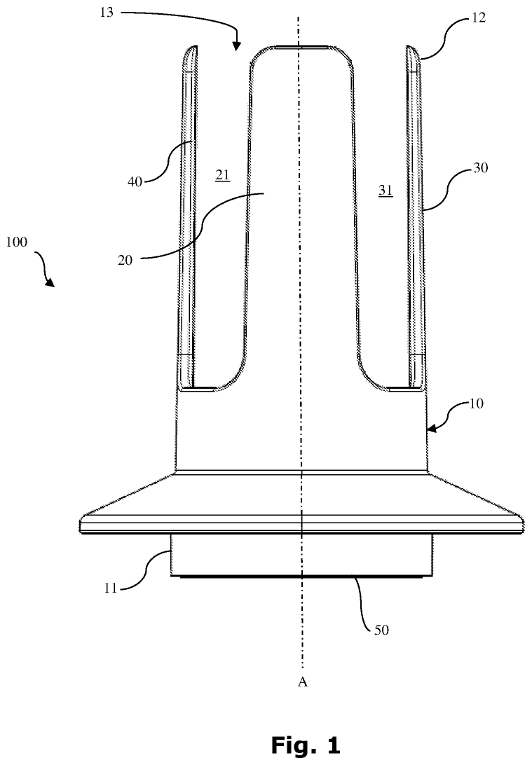

FIG. 1 depicts a side view of an anoscope according to a preferred aspect of the present disclosure;

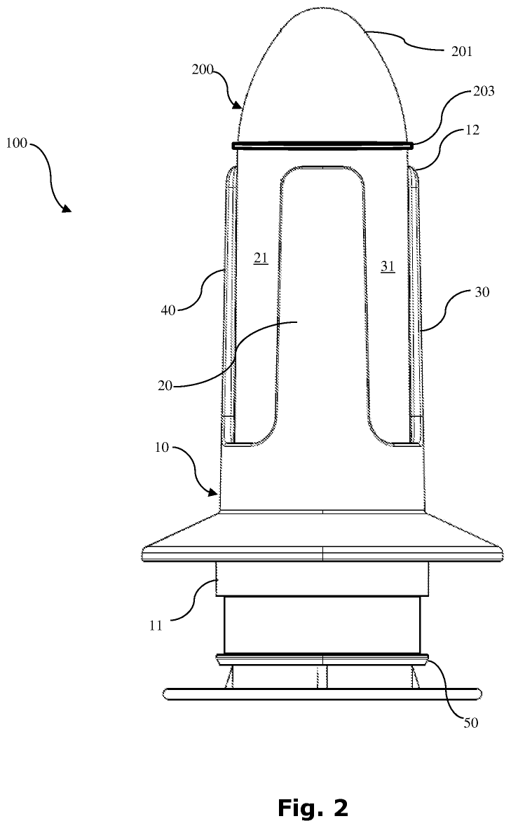

FIG. 2 illustrates a side view of an anoscope according to another preferred aspect of the present disclosure;

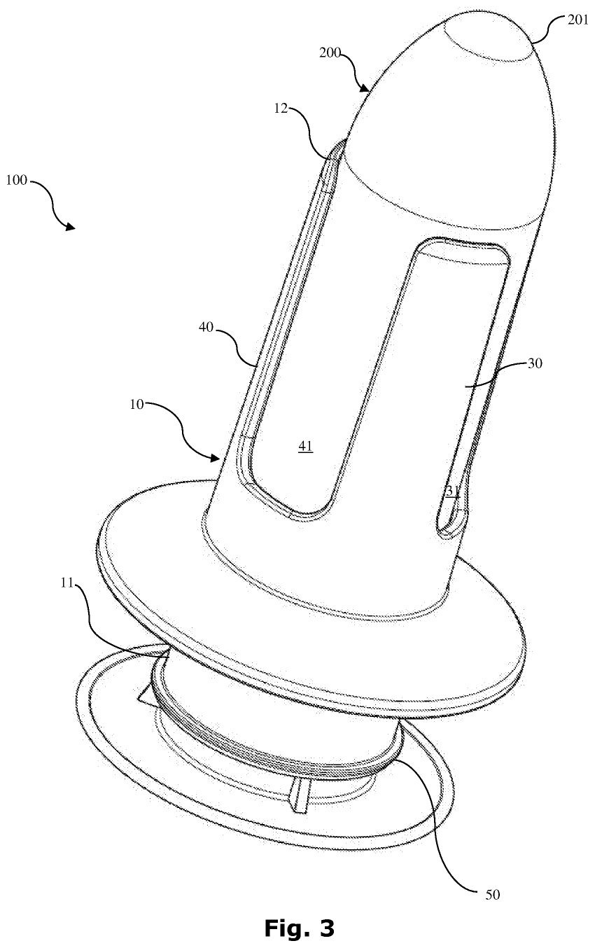

FIG. 3 shows a perspective view of an anoscope according to a preferred aspect of the present disclosure;

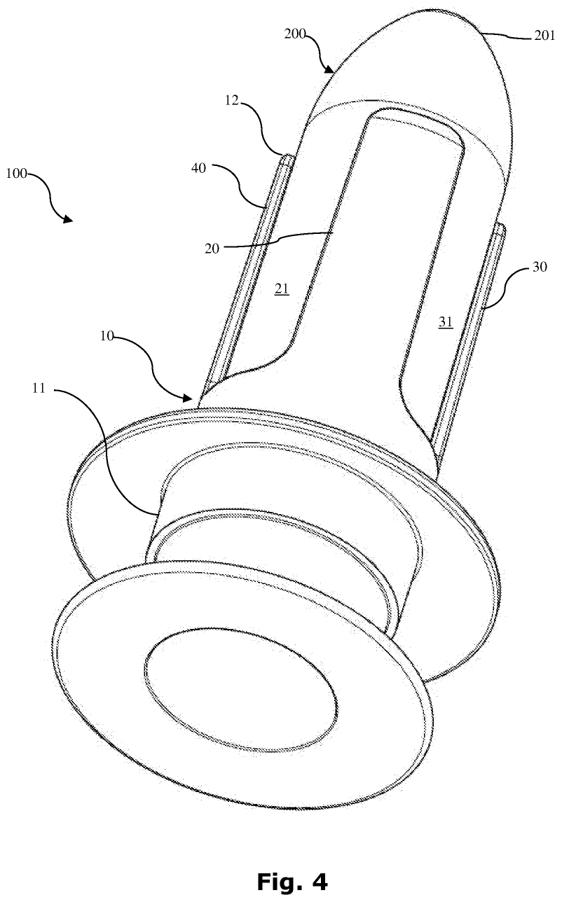

FIG. 4 displays another perspective view of an anoscope according to a preferred aspect of the present disclosure;

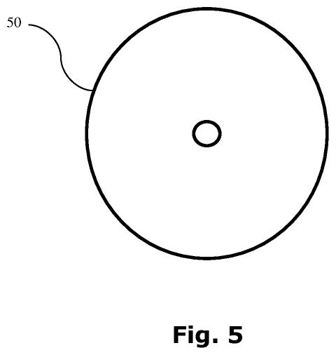

FIG. 5 presents a frontal view of a hermetic closure element of an anoscope according to a preferred aspect of the present disclosure;

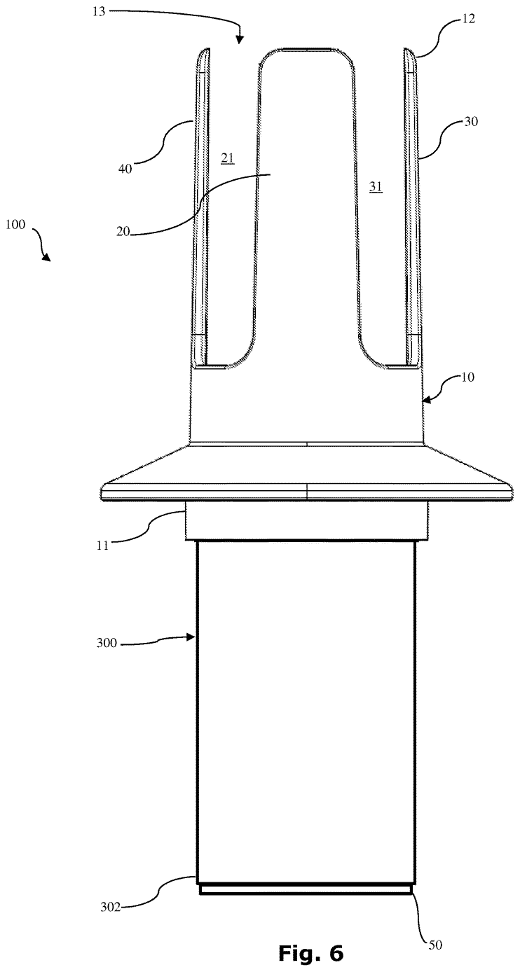

FIG. 6 illustrates a side view of an anoscope according to another preferred aspect of the present disclosure, in a first configuration;

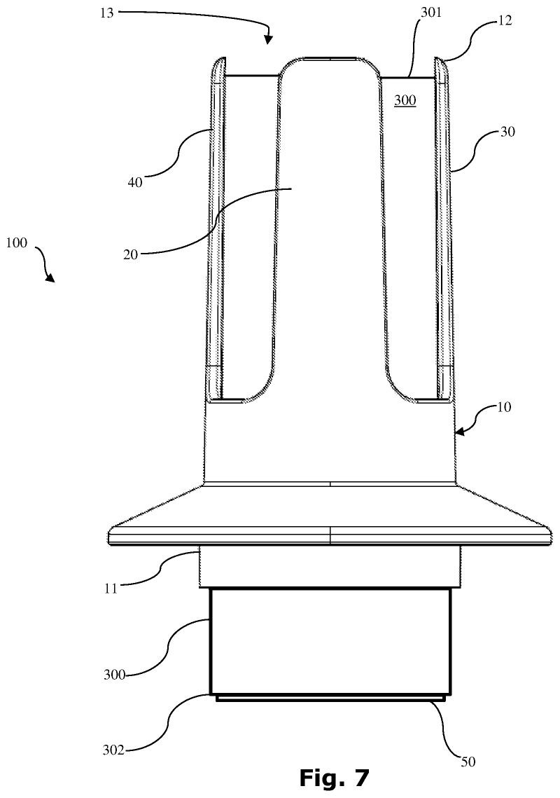

FIG. 7 shows a side view of an anoscope according to another preferred aspect of the present disclosure, in a second configuration;

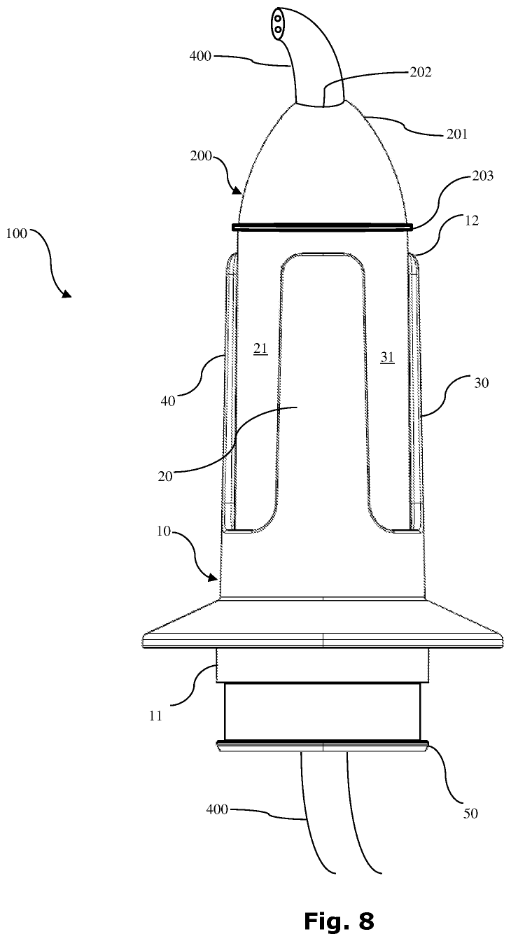

FIG. 8 displays a side view of an anoscope according to another preferred aspect of the present disclosure, in a fourth configuration;

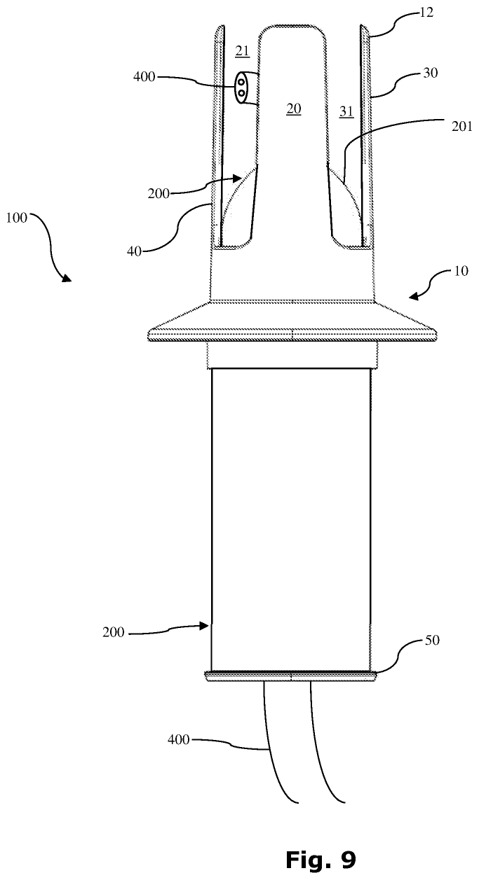

FIG. 9 depicts a side view of an anoscope according to another preferred aspect of the present disclosure, in a third configuration.

With reference to the attached figures, particularly FIG. 1, an embodiment of an anoscope is indicated by reference number 100.

The term “anoscope” within the context of this disclosure refers to a device configured for keeping the anal canal of a patient open to insert an endoscope for colonoscopy.

The anoscope 100 according to this disclosure comprises a main body 10. This main body 10 is configured to be inserted at least partially into the body of a patient, specifically into the anal canal, to keep it open during colonoscopy. Additionally, the main body 10 is configured, in use, to allow the passage of an endoscope for performing colonoscopy.

In more detail, the main body 10 extends along a main axis of development A between a first end portion 11 and a second end portion 12. This second end portion 12, opposite to the first end portion 11, is configured, in use, to be inserted at least partially into the body of a patient, particularly into the anal canal of a patient.

To allow, in use, the passage of an endoscope, the main body is configured to define a through cavity 13, precisely designed for the passage of an endoscope.

This through cavity 13 extends between the first end portion 11 of the main body 10 and the second end portion 12 of the main body 10.

In other words, the main body 10 is substantially a hollow body, presenting a through cavity in a longitudinal direction, i.e., parallel to the main axis of development A of the main body 10 itself, between its first end portion 11 and its second end portion 12. In this way, during colonoscopy, the main body 10 is configured to keep the anal canal of a patient open and, at the same time, allow the passage of an endoscope inside the patient's colon through the through cavity 13.

The main body 10 also includes a first surface, or outer surface, which is configured, in use, during colonoscopy, to face or come into contact with the anal canal of a patient. Opposite to this first surface, or outer surface, the main body 10 includes a second surface, or inner surface. In other words, this second surface, or inner surface, of the main body 10 is facing or directed towards the through cavity 13.

According to the present disclosure, the main body 10 also includes a plurality of openings 21, 31, 41. Specifically, each opening in the plurality of openings 21, 31, 41 is configured to connect the first surface, or outer surface, of the main body 10 with the second surface, or inner surface, of the same main body 10.

In other words, each opening in the plurality of openings 21, 31, 41 of the main body 10 is a slit, or gap, or window defined in the main body 10. In other words, this main body 10 is a fenestrated body.

Advantageously, the fact that the main body 10 of the anoscope 100 according to the present disclosure is a fenestrated body, or has openings, allows, during colonoscopy, direct visualization through these openings of at least a portion of a patient's anal canal.

Furthermore, the fact that these openings pass through the main body 10, connecting the outer surface to the inner surface of the main body 10, allows optimal visualization of a portion of a patient's anal canal without the visualized portion being compressed or flattened by the anoscope 100 that keeps the patient's anal canal open. This is particularly advantageous for the correct visualization of specific structures that may be present in the anal canal of some patients, such as hemorrhoids.

Finally, the fact that the main body 10 is a fenestrated body, or has openings, allows visualization of the distal rectum without having to perform the risky instrumental retroversion maneuver during colonoscopy.

Preferably, according to an aspect of the present disclosure, the main body 10 includes a plurality of protruding elements 20, 30, 40. Each protruding element of this plurality of protruding elements 20, 30, 40 extends from the first end portion 11 of the main body 10 toward the second end portion 12 of the same main body 10.

In other words, each protruding element in the plurality of protruding elements 20, 30, 40 is preferably an elongated body or element that protrudes from the first end portion 11 of the main body 10.

These protruding elements 20, 30, 40 are configured, on one hand, in use, to keep the anal canal of a patient open, defining the through cavity 13 or longitudinal opening of the main body 10, and on the other hand, to allow direct visualization, during colonoscopy, of at least a portion of the patient's anal canal, as these protruding elements 20, 30, 40 define, among them, said openings 21, 31, 41.

In particular, preferably, each opening in the plurality of openings 21, 31, 41 is defined between two consecutive protruding elements of the plurality of protruding elements 20, 30, 40.

For example, the main body 10 of the anoscope 100 according to the present disclosure may include three protruding elements 20, 30, 40 and three openings 21, 31,41.

According to this aspect, for instance, a first protruding element 20 and a second protruding element 30 define, between them, a first opening 21. Similarly, the second protruding element 30 defines, with a third protruding element 40, a second opening 31. Additionally, between the third protruding element 40 and the first protruding element 20, a third opening 41 is defined.

In order to keep, in use, the anal canal of a patient open and reduce the invasiveness of the colonoscopic examination, the protruding elements of the plurality of protruding elements 20, 30, 40 are preferably arranged circumferentially to form the through cavity 13 of the main body 10. In other words, the protruding elements 20, 30, 40 extend from the first end portion 11 toward the second end portion 12 of the main body 10 and are arranged circumferentially or around the main axis of development A of the main body 10.

Additionally, preferably, at least in the vicinity of the second end portion 12 of the main body 10, each protruding element in the plurality of protruding elements 20, 30, 40 has a chamfered, rounded, or curved configuration. Advantageously, in this configuration, the diagnostic examination can be less traumatic and less dangerous for a patient during use.

Additionally, preferably, the connecting portion between the first surface, or outer surface, and the second surface, or inner surface, of each protruding element in the plurality of protruding elements 20, 30, 40 is chamfered, rounded, or curved to reduce trauma and increase intervention safety. In other words, the peripheral edge of each protruding element 20, 30, 40 is chamfered, rounded, or rounded.

According to the present disclosure, the anoscope 100 further comprises a hermetic closure element 50. This hermetic closure element 50, visible, for example, in FIG. 5, is configured to hermetically seal the through cavity 13, particularly, preferably, at the first end portion 11 of the main body 10.

In this way, advantageously, in use, it is possible to create impermeability to the escape of insufflated gas during the endoscopic examination. This further results in less contamination for the operators.

Preferably, according to an aspect of the present disclosure, the hermetic closure element 50 is an element made of elastic material.

Even more preferably, this hermetic closure element 50 is made of silicone material.

Furthermore, according to a preferred aspect of the present disclosure, the hermetic closure element 50 includes a through hole. This through hole is configured, in use, for the passage of an endoscope. In this way, it is possible to perform a colonoscopy creating impermeability to the escape of insufflated gas during the examination. The elastic material of the hermetic closure element advantageously ensures impermeability around an endoscope during use. In combination or in alternative to the elastic material, the through hole may include a valve.

According to a further preferred aspect of the present disclosure, the anoscope 100 includes an actuation device. In other words, this actuation device is a manipulable element of the anoscope 100, directly operable by an operator performing a diagnostic examination using the anoscope 100, for example, during a colonoscopy. For example, the actuation device may consist of or include a handle, preferably operatively associated with the main body 10 of the anoscope 100. In other words, the actuation device may be an element protruding from the main body 10, preferably in a radial direction, and designed to be manipulated by an operator.

More specifically, preferably, this actuation device is associated with the first end portion 11 of the main body 10. Furthermore, the actuation device is configured, in use, to allow rotation of the main body 10 around its main axis of development A.

In this way, advantageously, during a colonoscopy examination, an operator can rotate the anoscope 100, and in particular the main body 10 around the main axis of development A, through the actuation device so that, through the apertures 21, 31, 41, a different portion of the anal canal wall of a patient can be visualized.

According to a further preferred aspect of the present disclosure, with particular reference to FIGS. 2 to 4, the anoscope 100 further comprises a dilator device 200. This dilator device 200 is configured to be removably inserted into the through cavity 13 of the main body 10, preferably along an insertion direction, preferably parallel to said main axis of development A.

Advantageously, the use of such a dilator device 200, for example, a stylet, particularly in the initial phase of a colonoscopy examination, especially during the insertion phase of the anoscope, allows a non-traumatic or less traumatic procedure for a patient.

In use, according to a preferred aspect of the present finding, the anoscope 100 is configured so that the dilator device 200 is inserted into the through cavity 13 of the anoscope 100 itself, which can then be inserted, as said, in a less traumatic way into the patient's anal cavity. Once the anoscope 100 is inserted, the dilator device 200 can be extracted or removed from the through cavity 13, and the hermetic closure element 50 can be placed in correspondence with the first end portion 11 of the main body 10 to hermetically seal the through cavity 13. Subsequently, it is possible to insert an endoscope, preferably through a through hole in the hermetic closure element 50, and perform the diagnostic examination.

According to a preferred aspect of the present disclosure, to prevent or reduce possible damage to the rectal tissue mucosa that may protrude into the apertures 21, 31, 41 towards the through cavity 13 of the main body, the anoscope 100 according to the present finding may include a cylindrical element 300.

This cylindrical element 300 is a substantially tubular element, comprising a through cavity that extends along the main axis of development A of the anoscope 100 and is configured to be inserted into the through cavity 13 of the latter, at least between a first position, or partially inserted position, visible, for example, in FIG. 6 and a second position, or fully inserted position, visible, for example, in FIG. 7.

In detail, in the second position, or fully inserted position, the cylindrical element 300 is configured, in use, to prevent the collapse of the rectal tissue of a patient into the apertures 21, 31, 41. This way, it is possible to rotate the anoscope 100 safely, thus avoiding possible damage to the rectal mucosa of the patient.

In use, once the anoscope 100 is rotated to visualize the rectal wall of the patient, it is possible to at least partially extract the cylindrical element 300 from the through cavity 13, bringing it into the first position, or partially inserted position, in which it is possible to directly visualize the rectal wall through the apertures 21, 31, 41.

If it becomes necessary to rotate the anoscope 100 again, for example, through the actuation device, to visualize another portion of the patient's anus, it is sufficient to insert the cylindrical element 300 into the second position, or fully inserted position, to allow the exit of potentially prolapsed mucosa into the apertures 21, 31, 41, and rotate the anoscope 100, and then return the cylindrical element 300 to the first position.

According to this aspect, to maintain the impermeability of the device, the cylindrical element 300 is hermetically insertable into the anoscope 100, particularly into the main body 10. To this end, a first gasket, preferably made of rubber, may be provided between the anoscope 100 and the cylindrical element 300.

Furthermore, still according to this aspect, to allow the insertion and sliding of the cylindrical element 300 into the through cavity 13 of the anoscope 100, the hermetic closure element 50 is advantageously positioned at a terminal portion of the cylindrical element 300, rather than at the first end portion 11 of the main body 10 of the anoscope 100. Specifically, the cylindrical element 300 comprises a first terminal portion 301 configured to be inserted into said main body 10, and a second terminal portion 302, opposite the first terminal portion, in which the hermetic closure element 50 is associated with the second terminal portion 302. In detail, this second terminal portion 302 of the cylindrical element 300 configured for coupling with the hermetic closure element 50 is a free terminal portion, or configured not to be inserted into the through cavity 13, and therefore into the main body 10, of the anoscope 100.

According to a further aspect of the present disclosure, the dilator device 200, like the cylindrical element 300, can be configured to avoid or reduce possible damage to the rectal tissue mucosa that may prolapse into the apertures 21, 31, 41, towards the through cavity 13 of the main body, and, at the same time, to allow effective visualization of the rectal tissue.

In particular, according to this aspect, the dilator device 200 is configured to be removably inserted into the through cavity 12 of the main body 10 of the anoscope 100, along an insertion direction, to allow a less traumatic insertion procedure of the anoscope 100 into the patient's body. To this end, preferably, the dilator device 200 may include at least a first end region 201, wherein this first end region 201 is tapered or substantially conical, or pointed. This first end region 201 is preferably configured to facilitate the insertion of the dilator device 200 itself, and consequently the anoscope 100, into the anal cavity of a patient, and to make this operation less traumatic for the patient.

Preferably, furthermore, the dilator device 200 may include a through-opening 202 located in correspondence with this first end region 201. Preferably, this through-opening 202 is located along the main axis of development A. In particular, this through-opening 202 is configured, in use, to allow the passage of an endoscope 400.

In a second end region of the dilator device 200, opposite the first end region 201, the hermetic closure element 50 can be provided, configured, in use, to hermetically seal the through cavity 12 and, at the same time, allow the passage of an endoscope 400. In other words, the hermetic closure element 50 is configured to be operatively associated with the second end region of the dilator device 200.

According to this aspect, preferably, the dilator device 200 is a hollow body having a circular or substantially circular section that extends along the main axis of development A of the anoscope 100 and is configured to be inserted into the through cavity 13 of the main body 10, at least between a third position, or partially inserted position, depicted, for example, in FIG. 9, and a fourth position, or fully inserted position, depicted, for example, in FIG. 8.

In this way, in the fourth position, or fully inserted position, the dilator device 200 is configured, in use, to prevent the collapse of the rectal tissue of a patient within the apertures 21, 31, 41, while in the third position, or partially inserted position, the dilator device 200 leaves at least partially free said apertures 21, 31, 41, allowing for the direct visualization of the rectal wall through such apertures 21, 31, 41.

Preferably, in order to prevent further movement of the dilator device 200 relative to the main body 10 beyond the third position, or partially inserted position, which would result in a separation of the dilator device 200 from the main body 10, the dilator device may include at least one protruding element 203, depicted exemplarily in FIG. 2, or in FIG. 8. This protruding element 203 is configured to prevent sliding of the dilator device 200 relative to the main body 10, at least along an extraction direction, opposite to said insertion direction, of the dilator device 200 from the through cavity 13 of the main body 10. Preferably, the protruding element 203 is an annular element, preferably located in proximity to the first end region 201 of the dilator device 200.

Furthermore, in order to maintain fluid tightness of the system, a second seal, preferably made of rubber, can be provided between the dilator device 200 and the main body 10 of the anoscope 100. Preferably, this second seal is arranged in the through cavity 13 of the main body 10. Also, preferably, this second seal can be configured to come into contact with the at least one protruding element 203 of the dilator device 200, thus preventing sliding of the dilator device 200 relative to the main body 10, at least along the extraction direction of the dilator device from the main body 10. In other words, this second seal serves as an end stop for the sliding of the dilator device 200 relative to the main body 10 along the extraction direction of the dilator device from the main body 10.

The present disclosure further relates to a diagnostic device comprising an endoscope 400 and an anoscope 100 as previously described.

The subject matter of the present disclosure has been described so far with reference to its embodiments. It is to be understood that other embodiments may exist that pertain to the same inventive core, all falling within the scope of protection of the claims set forth below.

1. An anoscope for colonoscopy, said anoscope comprising
a main body extending along a main axis of development between a first end portion of said main body and a second end portion of said main body, wherein said second end portion is configured, in use, to be inserted at least partially into the body of a patient, said main body defining a through cavity along said main axis of development between said first end portion and said second end portion of said main body,
an hermetic closure element configured to hermetically close said through cavity, wherein said main body of said anoscope includes a first surface, or outer surface, configured, in use, to face an anal canal of a patient, and a second surface, opposite to said first surface, or outer surface;
said main body comprising a plurality of openings each configured to connect said first surface, or outer surface, to said second surface, or inner surface, of said main body.

2. The anoscope according to claim 1, wherein said main body comprises a plurality of protruding elements, wherein each protruding element of said plurality of protruding elements extends from said first end portion of said main body toward said second end portion of said main body.

3. The anoscope according to claim 1, wherein each protruding element of said plurality of protruding elements is an elongated body protruding from said first end portion of said main body.

4. The anoscope (100) according to claim 1, wherein the protruding elements of said plurality of protruding elements are circumferentially arranged to form said through cavity of said main body.

5. The anoscope according to claim 4, wherein each opening of said plurality of openings is defined between two consecutive protruding elements of said plurality of protruding elements.

6. The anoscope according to claim 5, including three protruding elements and three openings.

7. The anoscope according to claim 1, wherein said hermetic closure element (50) is an element made of elastic material.

8. The anoscope according to claim 7, wherein said hermetic closure element (50) comprises a through-hole configured, in use, for passing an endoscope (400).

9. The anoscope according to claim 1, comprising a driving device associated with said first end portion (11) and configured to allow, in use, a rotation of said main body about said main axis of development (A).

10. The anoscope according to claim 1, comprising a dilator device configured to be removably inserted into said through cavity of said main body.

11. The anoscope according to claim 1, comprising a cylindrical element configured to be inserted into the through cavity of the main body, at least between a first position, or position of partial insertion, and a second position, or position of complete insertion.

12. The anoscope according to claim 11, wherein said cylindrical element is hermetically insertable into said main body.

13. The anoscope according to claim 12, wherein said cylindrical element comprises a first end portion configured to be inserted into said main body and a second end portion opposite said first end portion, wherein said hermetic closure element is associated with said second end portion.

14. The anoscope according to claim 10, wherein said dilator device comprises a first end region, said first end region being tapered or pointed.

15. The anoscope according to claim 14, wherein said dilator device comprises a through-opening located at said tapered end region; said through-opening being configured, in use, for the passage of an endoscope.

16. The anoscope according to claim 15, wherein said dilator device comprises a second end region opposite to said first end region, wherein said hermetic closure element is configured to be operatively associated with said second end region of said dilator device.

17. The anoscope according to claim 10, wherein said dilator device comprises at least one protruding element configured to prevent sliding of said dilator device relative to said main body at least along a direction of extraction of the dilator device from said through cavity of said main body.

18. A diagnostic device comprising an anoscope according to claim 1 and an endoscope (400).

19. The anoscope according to claim 14, wherein said dilator device comprises at least one protruding element configured to prevent sliding of said dilator device relative to said main body at least along a direction of extraction of the dilator device from said through cavity of said main body.

20. The anoscope according to claim 15, wherein said dilator device comprises at least one protruding element configured to prevent sliding of said dilator device relative to said main body at least along a direction of extraction of the dilator device from said through cavity of said main body.
